(19) United States
(12) Patent Application Publication
(10) Pub. No.: US 2003/0009111 A1
(43) Pub. Date: Jan. 9, 2003

(54) Non-invasive method and apparatus for tissue detection

(76) Inventors: Philip C. Cory, Bozeman, MT (US); Joan M. Cory, Bozeman, MT (US)

Correspondence Address: MCDERMOTT, WILL & EMERY, 600 13th Street, N.W., Washington, DC, 20005-3096, US

(21) Appl. No.: 10/170,194

(22) Filed: Jun. 13, 2002

Related U.S. Application Data
(60) Provisional application No. 60/297,694, filed on Jun. 13, 2001.

(51) Int. Cl. A61B 5/05

(52) U.S. Cl. 600/547

(57) ABSTRACT

An apparatus and method for non-invasively determining tissue structure by applying a periodic waveform to an external or internal body part. A microprocessor provides instructions to a waveform generator to generate a plurality of different periodic waveforms to at least one sampling electrode electrically connected to at least on return electrode through the tissue structure. The impedance of the tissue structures are selectively determined for each generated waveform. After determining a plurality of impedance measurements various calculations are performed, including determining a ratio of impedance change and the applied current change. The apparatus may apply the same waveform to all sampling electrodes simultaneously, or apply the waveform to a few as one sampling electrode at a time. The apparatus may also simultaneously apply a plurality of waveforms to a plurality of electrodes to maintain the same current waveform on each sampling electrode.

This application claims priority to U.S. Provisional application No. 60/297,694 filed on Jun. 13, 2001, herein incorporated by reference.

### FIELD OF THE INVENTION

The present invention relates to a non-invasive method and device for discriminating and mapping types of tissue. Particularly, the present invention relates to tissue discriminating and mapping by the application of a periodic waveform to a subject by monitoring induced changes in the electrical characteristics of the subject.

### BACKGROUND

Non-invasive detection of subcutaneous tissues has concerned many medical practitioners for many years. It is known by practitioners that many forms of subcutaneous tissue are responsive to electrical signals. Biologic, electrically responsive membrane systems (BERMS) are lipid bi-layers containing embedded protein molecules, some of which are ion channels. The density of embedded ion channels is known to show tissue type variability, with nerve tissue having the highest concentrations of ion channels per gram of tissue. Nerve abnormalities, such as neuromas, are known to have even higher concentrations of ion channels than normal nerve. Other tissues, such as muscle, have lesser amounts than normal nerve tissue.

BERMS are known to be responsive for electrical inductance in an externally applied electrical field. This membrane inductance is known to occur in addition to the widely appreciated membrane resistance and membrane capacitance. Subthreshold, alternating, electrical fields do not generate action potentials, but cause anomalous impedance (a reflection of the inductance), which has been noted and modeled in single axon systems. Mauro, ANOMALOUS IMPEDENCE, A PHENOMENOLOGICAL PROPERTY OF TIME-VARIANT RESISTANCE, AN ANALYTIC REVIEW, The Rockefeller Institute (1961), proposes a mechanism to explain this anomalous impedance, which is based on the effect of normal membrane currents flowing across the nerve cell membrane in the opposite direction to the applied field. These currents are associated with time variant, ion-specific conductance and behave, electrically, as inductance. In addition, Sabah and Leibovic, SUBTHRESHOLD OSCILLATORY RESPONSES OF THE HODGKIN-HUXLEY CABLE MODEL FOR THE SQUID GIANT AXON, Department of Biophysical Sciences, Center for Theoretical Biology, State University of New York at Buffalo, Amherst, N.Y. (1969), disclose circuit models of membrane electrical inductance, connected in parallel with membrane capacitance and membrane resistance and predict an electrical resonance effect.

Prior art for noninvasive determination of tissue depth, composition, configuration, and/or state of function from the skin surface either detects a change in the function of the structure in response to stimulation or assumes characteristics about electrical field paths in tissue. In one technique the location of nerve is detected by generating action potentials in nerves from certain electrodes within an array of electrodes.

U.S. Pat. No. 6,167,304 to Loos discusses the use of induced electrical fields to cause nerve “resonance”. It is unclear specifically what is meant by the term resonance in the Loos disclosure. This resonance occurs at certain frequencies and is associated with physiologic findings. However, it is clearly not the same as the electrical phenomenon of resonance, which is a function of inductance and capacitance connected either in series or in parallel and results in marked impedance changes at a single, unique frequency. The determination of impedance plays no role in the Loos resonance, which occurs at multiple frequencies.

U.S. Pat. No. 5,560,372 to Cory (herein incorporated by reference) teaches that, under certain conditions, the applied voltage required for maintenance of constant current flow through skin surface electrodes is reduced when measured on skin over the position of peripheral nerves as compared to skin not overlying significant nerve tissue. The device in Cory does not require action potential generation. This device indicated the lowest impedance site within its field by activating a single light emitting diode corresponding to the electrode contacting the skin surface at that site. This capability has not been addressed with other techniques, such as impedance tomography.

In the technique of impedance tomography, current flow between a pair of electrodes causes simultaneous voltage, amplitude, phase, or waveform variations at other electrodes arrayed on the body surface or in subcutaneous tissues which are not used to apply a current to the body surface, as described in U.S. Pat. No. 6,055,452 to Pearlman. Varying the electrode pairs through which current is flowing, followed by combining and analyzing the data, allows construction of specific impedance images of relevance to underlying structures. A key assumption for the performance of impedance tomography is that tissues have unique electrical characterizations, the most important being the specific impedance, tissue resistivity, and tissue dielectric constant. The electrical field itself supposedly does not affect these parameters, although changes in organ size, contents, conformation, or state of function are reflected in altered conductivity patterns. The technique of impedance tomography, above, analyze voltage information from the skin surface at points distinct from the stimulating pair of electrodes. The assumption is made that tissue resistivities or dielectric constants are stable in the presence of these electrical fields, allowing the calculation of current flow patterns beneath the skin surface and construction of images from those patterns. In this technique, resolution of subsurface structures remains a problem.

Accordingly, there exists a need to non-invasively detect tissue substructures in a sample which can accurately locate and discriminate the tissue substructures.

### SUMMARY OF THE INVENTION

The present invention provides an apparatus and method of accurately locating and discriminating tissue substructures which avoids the problems of the prior art.

An apparatus of the present invention may comprise: a microprocessor; a waveform generator operable to generate a plurality of different periodic waveforms in response to instructions received from the microprocessor; at least one sampling electrode operable to receive a waveform from the waveform generator and to apply the received waveform to a tissue of the subject as an applied waveform; at least one return electrode operable to receive the applied waveform from the tissue of the subject and to provide the applied waveform to the microprocessor, thereby completing an electrical circuit which includes the tissue of the subject as a component, wherein the microprocessor receives information indicative of the voltage and current of the applied waveform and calculates a non-linear electrical characteristic of the tissue of the test subject.

In the apparatus of the present invention, the non-linear characteristic which is calculated may be the impedance and/or the reactance of the tissue.

In the apparatus of the present invention, the microprocessor may be operable to: instruct the waveform generator to generate a plurality of different waveforms to be applied to the tissue, to selectively calculate the impedance of the tissue for each generated waveform of the plurality of different waveforms, and to determine a ratio of a change in impedance to a change in applied current.

In the apparatus of the present invention the at least one sampling electrode may comprise a plurality of sampling electrodes and the apparatus may further comprise a switching device operable to receive instructions from the microprocessor to provide a waveform to any sampling electrode of the plurality of sampling electrodes.

In the apparatus of the present invention, the switching device may be operable to simultaneously provide a single waveform to more than one sampling electrode.

In the apparatus of the present invention, the switching device may be operable to simultaneously provide a plurality of waveforms to more than one sampling electrode in a manner which provides the same current waveform to each of the sampling electrodes of the more than one sampling electrode.

In the apparatus of the present invention, the at least one return electrode may comprise a plurality of return electrodes and wherein the apparatus further comprises a return switching device operable to receive instructions from the microprocessor to select any return electrode of the plurality of return electrodes to thereby complete an electrical circuit between the at least one sampling electrode and the selected return electrode.

In the apparatus of the present invention, the at least one sampling electrode may comprise a plurality of sampling electrodes and the apparatus may further include a switching device operable to receive instructions from the microprocessor to provide a waveform to any sampling electrode of the plurality of sampling electrodes, and the at least one return electrode may comprise a plurality of return electrodes and the apparatus may further include a return switching device operable to receive instructions from the microprocessor to select any return electrode of the plurality of return electrodes to thereby complete an electrical circuit between the at least one sampling electrode and the selected return electrode.

The apparatus of the present invention may further comprise a display, and the microprocessor may generate a three dimensional image of the tissue and the display may be operable to display the three dimensional image.

The method of detecting tissue structures of the present invention may comprise the steps of: generating a periodic waveform; providing the periodic waveform to tissue of a subject through at least one sampling electrode as an applied waveform; receiving the applied waveform from the tissue of the subject through at least one return electrode, thereby completing an electrical circuit which includes the tissue of the subject as a component, receiving information indicative of the voltage and current of the applied waveform; and calculating a non-linear electrical characteristic of the tissue of the test subject associated with the applied waveform.

In the method of the present invention, the non-linear characteristic which is calculated may be the impedance of the tissue and/or the reactance of the tissue.

The method of the present invention may further comprise the steps of: generating a new periodic waveform which is different from a previous periodic waveform, providing the new periodic waveform to the tissue of a subject through the sampling electrode as another applied waveform; receiving the another applied waveform from the tissue of the subject through the return electrode, thereby completing an electrical circuit which includes the tissue of the subject as a component, receiving information indicative of the voltage and current of the another applied waveform; and calculating a non-linear electrical characteristic of the tissue of the test subject associated with the another applied waveform.

In the method of the present invention, the non-linear electrical characteristic which is calculated may be the impedance of the tissue, and the recalculated non-linear electrical characteristic may be the impedance of the tissue, the method may further comprise the step of performing mathematical calculations selectively using characteristics of the another applied waveform and characteristics of the applied waveform and the calculated impedance of the tissue and the recalculated impedance of the tissue.

In the method of the present invention, the mathematical calculation that is performed may be a determination of a ratio of a change in impedance to a change in applied current.

In the method of the present invention the at least one sampling electrode may comprise a plurality of sampling electrodes, and wherein the method further comprises the step of: simultaneously providing a single waveform to more than one sampling electrode.

The method of the present invention may further comprise the steps of: generating a new periodic waveform which is different from a previous periodic waveform, providing the new periodic waveform to the tissue of a subject through the sampling electrode as another applied waveform; receiving the another applied waveform from the tissue of the subject through the return electrode, thereby completing an electrical circuit which includes the tissue of the subject as a component, receiving information indicative of the voltage and current of the another applied waveform; and calculating a non-linear electrical characteristic of the tissue of the test subject associated with the another applied waveform.

The method of the present invention may further comprise the steps of: calculating the impedance of the tissue for the new periodic waveform, and determining a ratio of a change in impedance and a change in applied current determined for the tissue of the test subject for the applied waveform and the another applied waveform.

In the method of the present invention the at least one sampling electrode may comprise a plurality of sampling electrodes, and the method may further comprise the step of: simultaneously providing a plurality of waveforms to more than one sampling electrode in a manner which provides the same current waveform to each of the sampling electrodes of the more than one sampling electrode.

The method of the present invention may further comprise the steps of: generating a three dimensional image display of the tissue; and displaying the three dimensional image.

A computer readable medium embodying the present invention may carry instructions to cause a computer to institute the performance of a method, the method comprising the steps of: generating a periodic waveform; providing the periodic waveform to tissue of a subject through at least one sampling electrode as an applied waveform; receiving the applied waveform from the tissue of the subject through at least one return electrode, thereby completing an electrical circuit which includes the tissue of the subject as a component, receiving information indicative of the voltage and current of the applied waveform; and calculating a non-linear electrical characteristic of the tissue of the test subject associated with the applied waveform.

### BRIEF DESCRIPTION OF THE DRAWINGS

The accompanying drawings, which are incorporated in and form a part of the specification, illustrate the various embodiments of the invention and, together with the description, serve to explain the principles of the invention. In the drawings:

FIG. 1 illustrates the effect of an applied electric field in an ideal homogeneous medium;

FIG. 2 illustrates the relationship between current and voltage in an applied electric field in a homogeneous medium;

FIG. 3 illustrates the relationship between impedance and electrode separation distance for a fixed frequency of an applied electric field;

FIG. 4 illustrates the relationship between impedance and electrode separation distance for a fixed frequency higher than that in FIG. 3;

FIG. 5 illustrates a tissue detection apparatus according to a first embodiment of the present invention;

FIG. 6 illustrates a method of detecting tissue structures which may be used with the first embodiment of the present invention;

FIG. 7 illustrates another method of detecting tissue structures which may be used with the first embodiment of the present invention;

FIG. 8 illustrates yet another method of detecting tissue structures which may be used with the first embodiment of the present invention;

FIG. 9 illustrates still another method of detecting tissue structures which may be used with the first embodiment of the present invention;

FIG. 10 illustrates a second embodiment of the present invention; and

FIG. 11 illustrates a third embodiment of the present invention.

### DETAILED DESCRIPTION OF THE INVENTION

Reference will now be made in detail to the present preferred embodiments of the invention, an example of which is illustrated in the accompanying drawings.

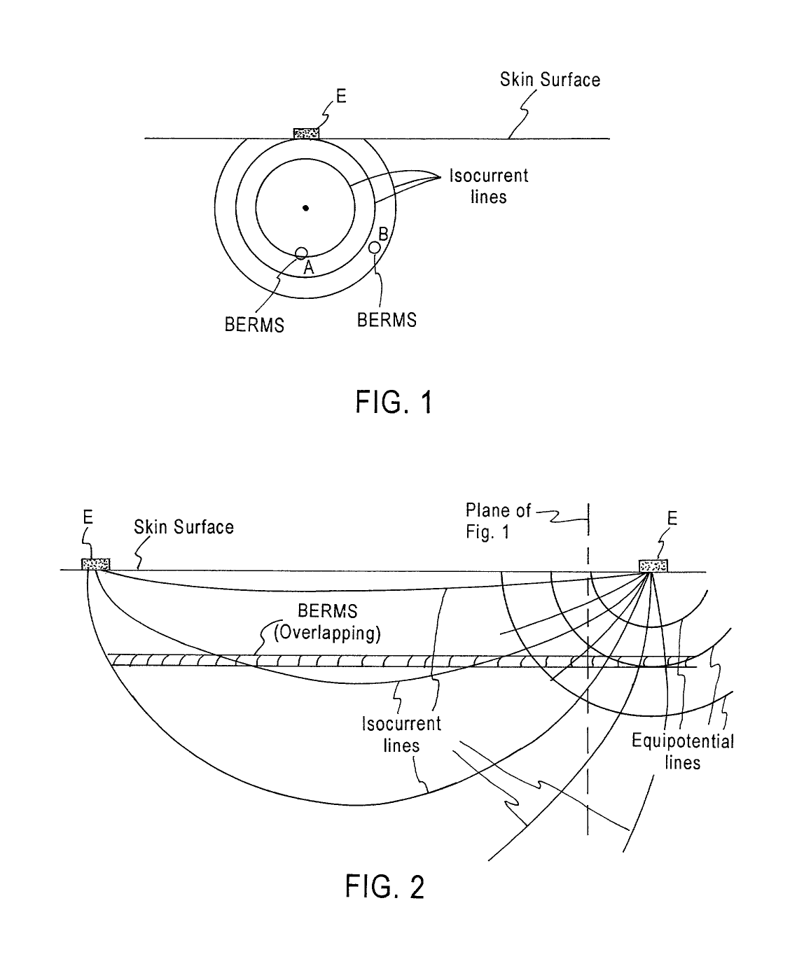

The inventors of the present invention made observations consistent with inductances that occur in the cell membrane affecting measurements performed over tissues. It has been further observed that (a) tissue resistivity and dielectric constants display negative, non-linear relationships to variable, increasing currents and (b) a resonance phenomenon often results from the interaction of the membrane-associated inductance and a membrane-associated capacitance. FIGS. 1-2 are directed to discussions with a homogeneous medium to illustrate the principle of operation of the invention. However, as those of skill in the art will appreciate that most living tissue is non-homogeneous, the present invention is directed toward detection of tissues in a non-homogeneous as well as homogeneous tissue.

With regard to (a) above, as illustrated in FIGS. 1 and 2, the scalar quantity current (or electrical intensity) follows a spindle shaped distribution between two skin surface electrodes. FIG. 1 illustrates the current distribution in a homogeneous medium. The current density at a point farther away from the center of the current distribution spindle will be lower than the current density closer to the center of the current distribution spindle. In a homogeneous medium, as illustrated in FIG. 1, concentric rings of isocurrent lines are formed in planes intersecting the line of the current-carrying electrodes at 90°. Thus, BERMS A is located on an isocurrent line having a higher current density than BERMS B. The actual current density at BERMS B will be lower that at BERMS A. As illustrated in FIG. 2, in a homogeneous medium, the voltage distributions will be substantially hemicircular about the skin surface electrodes with the equipotential lines at right angles to the isocurrent lines.

In a non-homogeneous medium, subsurface structures arrayed along an individual equipotential line will experience different actual current densities depending on their distance from the center of the current distribution spindle. This means that, in a non-homogeneous medium, the resistivity and dielectric constants of identical tissues will vary depending on the distance a measurement point lies from the center of the current distribution spindle. Alterations in applied current (I) occurring at the skin surface will cause the measured impedance (Z) at any point in the electrical field to change as a consequence of the resistivity variations induced by current density shifts at that particular measurement point.

It is generally known in the art that impedance Z contains a resistance component R and a reactive component (reactance) X, e.g. Z=R+jX, where j represents the imaginary operator (the square root of −1). The resistive component is often labeled as the “real” part of the impedance and the reactive component is often labeled as the “imaginary” part of the impedance. Resonance occurs when the inductive reactance and capacitive reactance are equal, and when the critical frequency=1/(2π√(LC)). If the inductance and capacitance are in parallel, at the critical frequency, Z [symbol] ∞; if the inductance and capacitance are in series, at the critical frequency, Z [symbol] 0. The field may have a frequency, in which case, the reactance cannot be zero since the capacitive reactance X_{c}=½πfC, and the inductive reactance X_{L}=2πfL. The loss of the reactive component may occur in two situations: when f [symbol] 0, X [symbol] 0 or when f [symbol] ∞, X [symbol] 0. The inventors have discovered that for a specified waveform and distance between the sampling electrode and the return electrode, various types of tissues may be identified and discriminated by observing BERMS-related changes in impedance.

In FIGS. 1 and 2, an electrode (E) is located on an ideal skin surface over ideal, homogeneous subcutaneous tissue. In FIGS. 1 and 2, two ideal, identical BERMS are located the same distance beneath the skin surface, one at a normal angle to the position of E (A) and the other at an angle <90° to E (B). For an electrical field at 90° to the plane connecting the two BERMS and the skin surface electrode, A will experience a greater current density than B. (It is recognized that the shape of the current density distribution will be altered by the BERMS in the real situation, but for discussion purposes, this effect will be ignored.) This will be true for all applied current levels and means that the ΔZ/ΔI will be greater for A than for B.

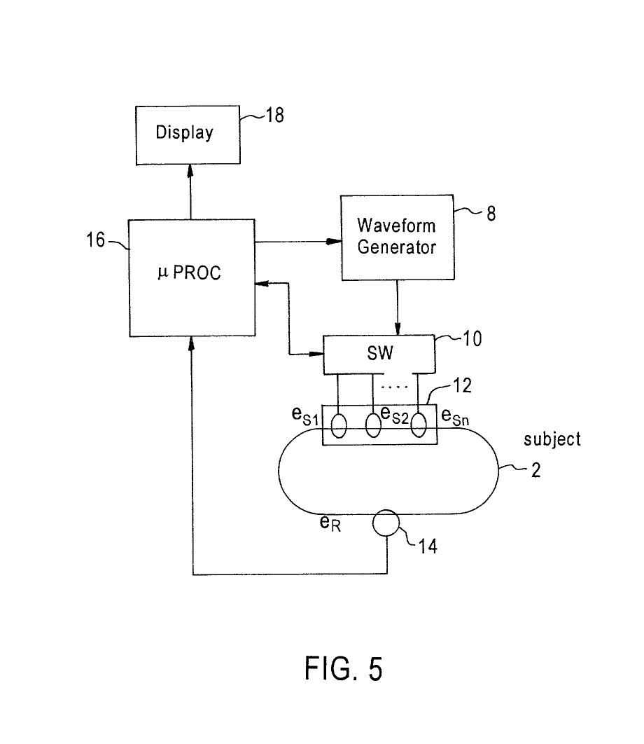

FIG. 5 illustrates a block diagram of an apparatus for detecting impedance changes associated with BERMS in either a homogeneous or non-homogeneous tissue in accordance with a first embodiment of the invention. As illustrated in FIG. 5, sample electrode array 12 is attached to a test subject 2 and return electrode 14 is also attached to the test subject 2 a distance d away from the sample electrode array 12. The test subject may be any tissue, including an external body part such as an arm, or an internal organ of a being. The test subject preferably contains at least one electrically responsive membrane system (a BERMS) comprising a lipid bi-layer containing embedded protein molecules, some of which are ion channels. The sampling electrode array 12 preferably comprises a sampling electrode having an array of a plurality of sample electrodes eₛ₁ through eₛₙ. Each of the sampling electrodes is preferably provided with an aqueous interface for making good electrical contact with the surface of subject 2.

Referring to FIG. 5, a current source preferably provides a current to waveform generator 8. A microprocessor 16 provides instructions to the waveform generator 8 to generate a periodic current waveform. The waveform generated by waveform generator 8 is preferably provided to switching device 10. The switching device 10 is preferably controlled by the microprocessor 16 to provide the generated waveform to a selected sample electrode eₛ₁ through eₛₙ for a predefined period of time (a sampling period). In the preferred embodiment, the waveform generator may control and change the amplitude, the frequency and the shape of the waveform generated, such as generating a pulsed train waveform, a sinusoidal waveform, a sawtooth waveform, etc. Alternatively, the microprocessor 16 may instruct the waveform generator 8 and switching device 10 to apply a plurality of different waveforms, each waveform being applied within a sampling time, to an individual sampling electrode prior to switching to another sampling electrode.

The switching device 10 may be a multiplexer or a gate array or any suitable device that may be controlled by the microprocessor 16 to provide current from the waveform generator 8 to the sampling electrode array 12. In the preferred embodiment, the switching device 10 may be controlled by the microprocessor 16 to apply the generated waveform to a single sampling electrode or to all or part of the sampling electrodes simultaneously. The waveform generator 8 may also be controlled by the microprocessor in association with the switching device 10 to apply the same current to a plurality of sampling electrodes or all of the sampling electrodes independently of each other simultaneously, even when the sampling electrodes experience different impedances. The waveform generator 8 and the switching device 10 may also be controlled by the microprocessor to apply a single current to all of the sampling electrodes or a plurality of sampling electrodes of the sampling electrode array so that the single current is dispersed among the selected sampling electrodes. With software control of the waveform, the current can be varied at an individual sample electrode within the array of electrodes, either during one sampling session or after sampling the other electrodes in the array.

The microprocessor 16 may be any type of computing device. In the preferred embodiment, the microprocessor 16 is programmed with software that allows the microprocessor to receive commands from an operator to define the parameters of the waveform, such as the shape of the waveform, the positive and negative peak amplitudes, the frequency and the duty cycle. The microprocessor may also contain a memory bank having a plurality of predefined waveforms and may select waveforms to be generated by the waveform generator from the predefined set of waveforms. The waveforms may change in positive peak amplitude, negative peak amplitude, frequency, shape, and/or duty cycle.

Still referring to FIG. 5, the return electrode 14 completes an electrical circuit with the sampling electrode array 12, allowing current to pass through the sampling electrode. In the preferred embodiment, the microprocessor detects a current during the sampling time (the period in which a waveform is applied to a sample electrode). The microprocessor preferably calculates and stores an impedance value for a plurality of sampling periods, during which a plurality of different waveforms are applied to the sampling electrode. In the preferred embodiment, the microprocessor 16 receives information from switching device 10 relating to the current waveform and the voltage waveform present at each sample electrode. The microprocessor preferably uses the current waveform and the voltage waveform at each sampling electrode to calculate the impedance between each sample electrode and the return electrode 14. The microprocessor preferably includes storage capability, such as a RAM, or a recordable magnetic, optical, or magneto-optical disk device, or a tape storage device. The microprocessor preferably stores data indicative of the current waveform, the voltage waveform and the calculated impedance for each sample electrode and for each sample period.

When ΔZ/ΔI is determined for all the electrodes in the array, those electrodes demonstrating the greatest ΔZ/ΔI will most directly overlie the course of the BERMS structure (e.g., a nerve) or have the largest quantity of BERMS (e.g., a nerve branch point) underlying those electrodes.

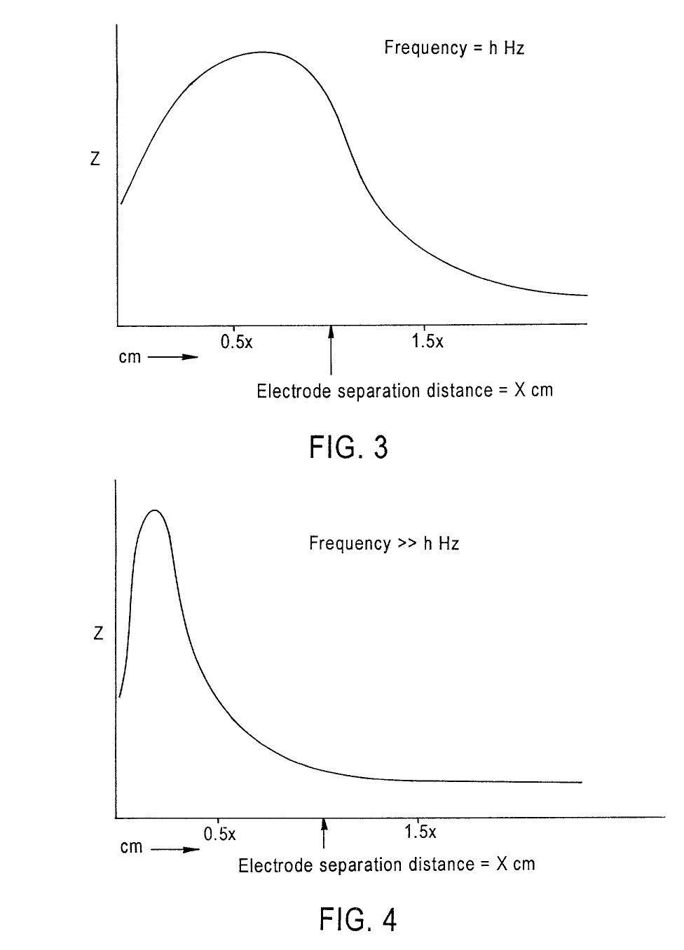

The frequency of the applied electrical field may be similarly varied to manipulate resonant peaks. As an example, in FIGS. 3 and 4, a nerve is composed of multiple, parallel electrical elements, the axons. Each axonal cell membrane is a BERMS. For a defined separation distance between the sampling electrode and the return electrode, each axon will have a specific resonant frequency. The impedance changes observed between the sampling electrode 12 and the return electrode 14 reflect all axonal resonance and give a broad impedance peak over a range of frequencies. Conversely, if a stable frequency is maintained and the distance d between the sampling electrode 12 and the return electrode 14 is varied, a broad peak will be seen over a range of separation distances, as illustrated in FIG. 3. An impedance peak may be eliminated at a specific electrode separation distance d, by increasing the frequency of the applied electrical field significantly above the resonant frequencies (FIG. 4). The ΔZ/ΔI effects then become a greater percentage of the overall impedance, maximizing their detection. Conversely, by lowering the frequency of the electrical field to broaden the impedance peak, examination of the individual components of the impedance peak with Fourier analysis, or similar mathematical approaches, is facilitated. In this manner, the operator may be able to focus on desired tissue structures.

In a first embodiment of the method of the invention, after the lapse of the sampling period, the microprocessor 16 preferably instructs the switching device 10 to provide the generated waveform to another sample electrode, such as eₛ₂ for the sampling time. The generated waveform is preferably provided to each sampling electrode in a sampling cycle in a predefined order. At the end of the sampling period, the microprocessor preferably instructs the waveform generator 8 to generate a different waveform to be applied to the sampling electrode array 12.

The impedance of the tissue structures are selectively determined for each generated waveform, i.e. the operator may provide instructions to avoid determining the impedance for some of the generated and applied waveforms. After determining a plurality of impedance measurements various mathematical analyses are performed using the plurality of impedance measurements, including determining a ratio of impedance change and the applied current change. The mathematical analyses may also consist of any effective data presentation technique, including but not limited to: raw data, normalization of raw data, rates of change between neighboring electrodes, use of rolling averages, presentation of percentage difference, or more complex analyses such as Fourier analysis of frequency components.

The microprocessor may also determine the individual components of the impedance measurement, e.g. the resistance and the reactance. The resistance and reactance may be calculated using known techniques, such as using a Fourier analysis technique to obtain the real (resistive) and imaginary (reactance) components of the impedance.

The microprocessor preferably provides a display signal to display 18. The microprocessor may generate two dimensional and three dimensional images, such as a three-dimensional topographic image, of the tissue structure to be displayed on the display 18. The generation of the two dimensional and three dimensional images may performed by using the plurality of impedance measurements with different waveforms. For example, directly measured values, or calculated results based on measured values, may be assembled into an image consisting of a single line, a two-dimensional topographic display, or a three-dimensional display of tissue and nerve contents.

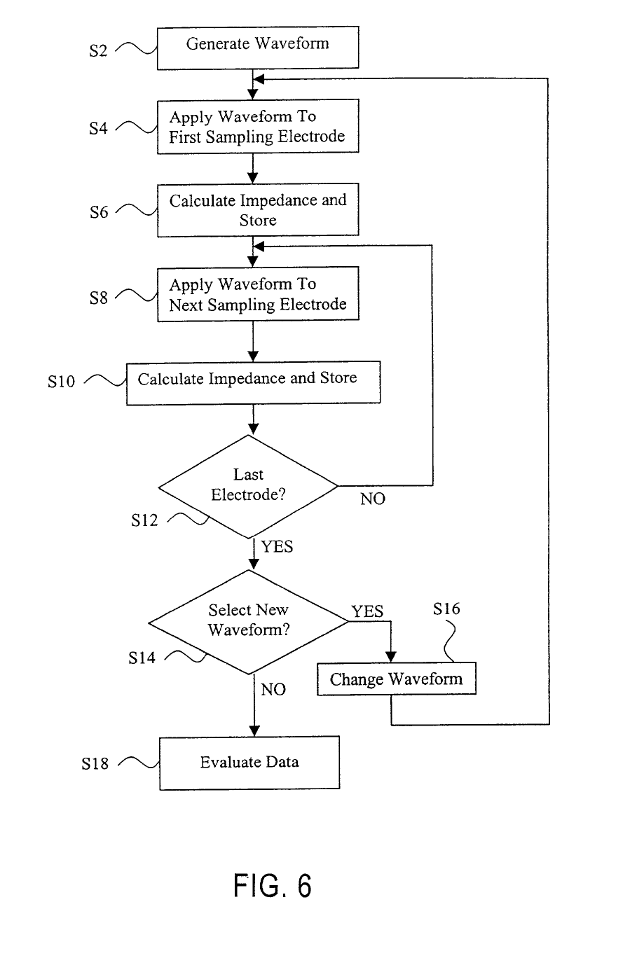

FIG. 6 illustrates a flow diagram of the first embodiment of a method of operating the apparatus of FIG. 5. As illustrated in FIG. 6, a waveform is generated (step S2) and applied to the first sampling electrode (step S4) during a sampling period. The impedance is calculated based on the characteristics of the applied waveform at the selected sampling electrode, such as voltage, current, frequency, and duty cycle ect., and the characteristics and the calculated impedance are stored by the microprocessor (step S6). The waveform is applied to another sampling electrode (step S8), which is preferably selected by switching device 10. The impedance is calculated again based on the characteristics of the applied waveform at the newly selected sampling electrode and the characteristics and the calculated impedance are stored by the microprocessor (step S1 ). The apparatus applies the waveform to each of the sampling electrodes by repeating steps S8 and S10 until the waveform has been applied to the last sampling electrode (step S12, NO). Once the waveform has been applied to all of the sampling electrodes (step S12, YES), the apparatus determines if there is another waveform to select (step S14) by determining if there are any waveforms in a predefined set of waveforms which have not been applied to the sampling electrodes or by prompting the operator to select another waveform. The new waveform may be changed from the previous waveform in maximum or minimum amplitude, in shape of the waveform, and/or in frequency or duty cycle. If another waveform is selected (step S14, YES), the waveform generator 8 generates a new waveform and applies it to the first sampling electrode S4. Steps S4-S12 are repeated with the new waveform. Once all of the waveforms have been applied to the sampling electrodes (step S14, NO), the microprocessor 16 evaluates the data by various mathematical calculations. For example, the microprocessor may determine the ΔZ/ΔI from the stored impedance, and the voltage and current data for each sampling electrode when applied with each waveform (step S18). The microprocessor may also determine the reactance of the tissue. In the preferred embodiment the operator may be able to instruct the microprocessor to perform any type of calculation.

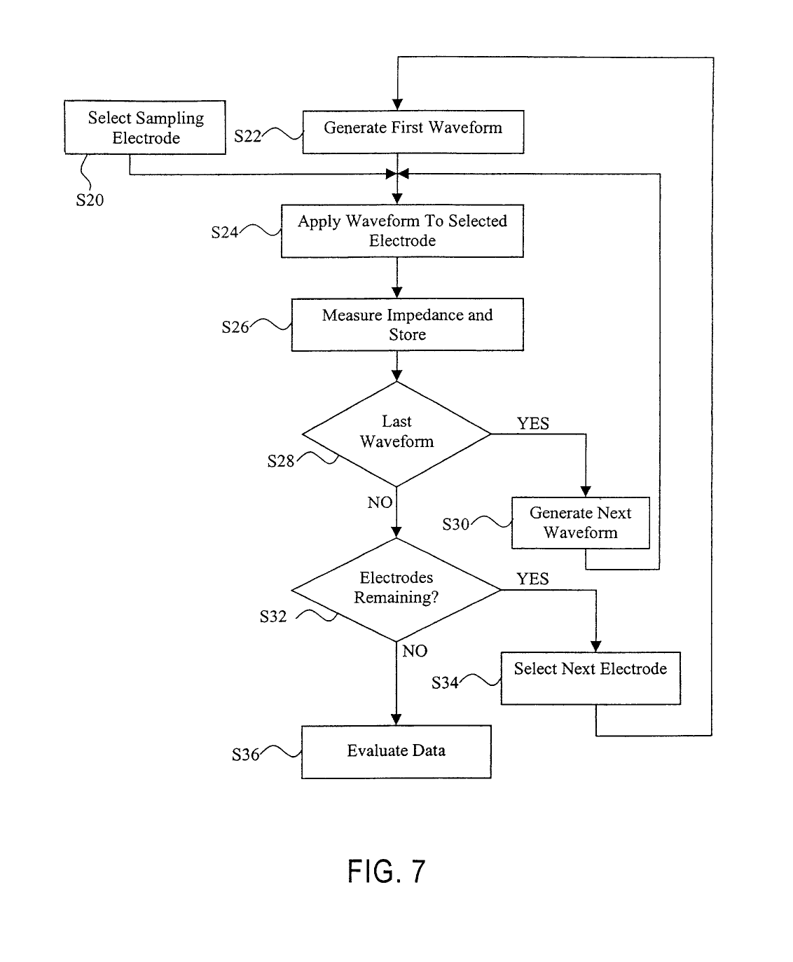

An alternative method is illustrated in FIG. 7. As illustrated in FIG. 7, a sampling electrode is selected (step S20) and a waveform is generated (step S22) and applied to the selected sampling electrode (step S24). The impedance is calculated based on the characteristics of the applied waveform at the selected sampling electrode, such as voltage, current, frequency, and duty cycle ect., and the characteristics and the calculated impedance are stored by the microprocessor (step S26). In step S28, the apparatus determines if there is another waveform to select (step S28) by determining if there are any waveforms in a predefined set of waveforms which have not been applied to the sampling electrodes or by prompting the operator to select another waveform. The new waveform may be changed from the previous waveform in maximum or minimum amplitude, in shape of the waveform, and/or in frequency. If another waveform is selected (step S28, YES), the waveform generator 8 generates a new waveform (step S30) applies it to the selected sampling electrode (steps S24 and S26). If no more waveforms are selected (step S28, NO), the apparatus determines if there are any sampling electrodes remaining which have not be applied with a the plurality of waveforms (step S32). If there are sampling electrodes remaining to be selected (step S32, YES), then a remaining sampling electrode is selected and the plurality of waveforms are applied to the newly selected electrode repeating steps S22-S30. If there are no sampling electrodes remaining (step S32, NO), the microprocessor 16 evaluates the data by various mathematical calculations. For example, the microprocessor may determine the ΔZ/ΔI from the stored impedance, voltage and current data for each sampling electrode when applied with each waveform (step S18). The microprocessor may also determine the reactance of the tissue. In the preferred embodiment the operator may be able to instruct the microprocessor to perform any type of calculation.

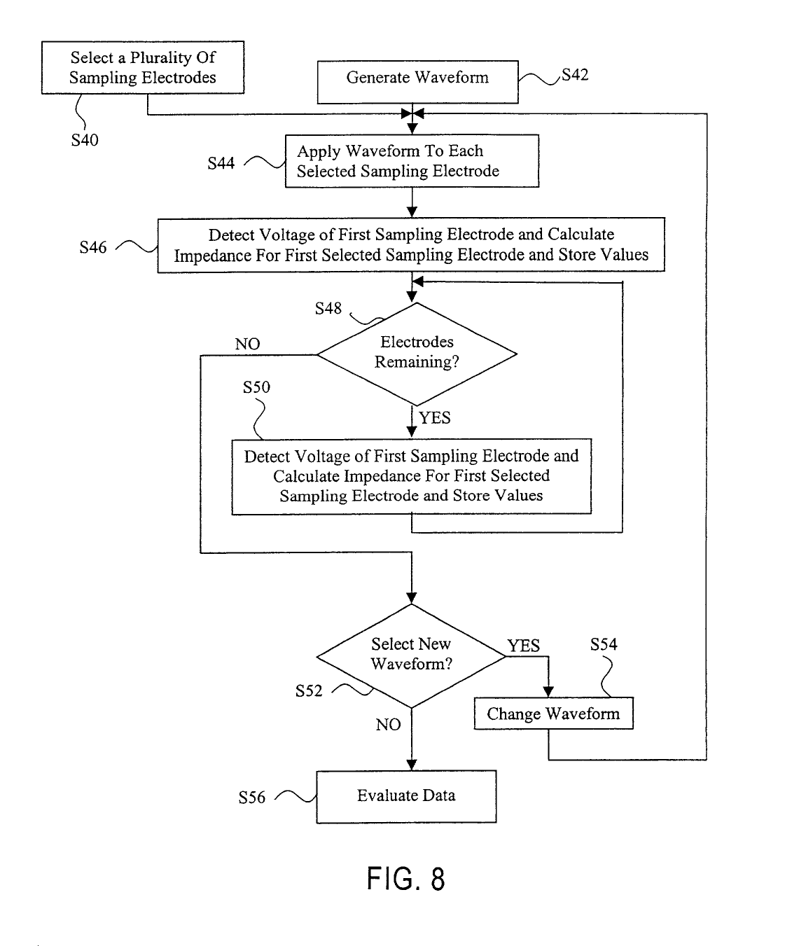

FIG. 8 illustrates another method according to the present invention. As illustrated in FIG. 8, a plurality of sampling electrodes are selected (step S40) a generated waveform (step S42) is applied to each of the selected sampling electrodes in a manner so that each selected electrode receives the same current waveform (step S44). The voltage of each selected sampling electrode is detected and the impedance of each of the selected sampling electrodes is determined (steps S46, S48 and S50). Since each of the selected sampling electrodes are applied with the same current, the voltage may vary between each of the sampling electrodes, thus the voltage is the only unknown variable needed to determine the impedance. Once the impedance is determined for the selected sampling electrodes (step S48, NO), the flow diagram determines if another waveform is to be selected (step S52). If a new waveform is to be selected, a new waveform is generated (step S54), applied to the selected sampling electrodes, and steps S44-S52 are repeated. If a new waveform is not selected, the microprocessor 16 evaluates the data by various mathematical calculations. For example, the microprocessor may determine the ΔZ/ΔI from the stored impedance, voltage and current data for each sampling electrode when applied with each waveform (step S56). The microprocessor may also determine the reactance of the tissue. In the preferred embodiment the operator may be able to instruct the microprocessor to perform any type of calculation.

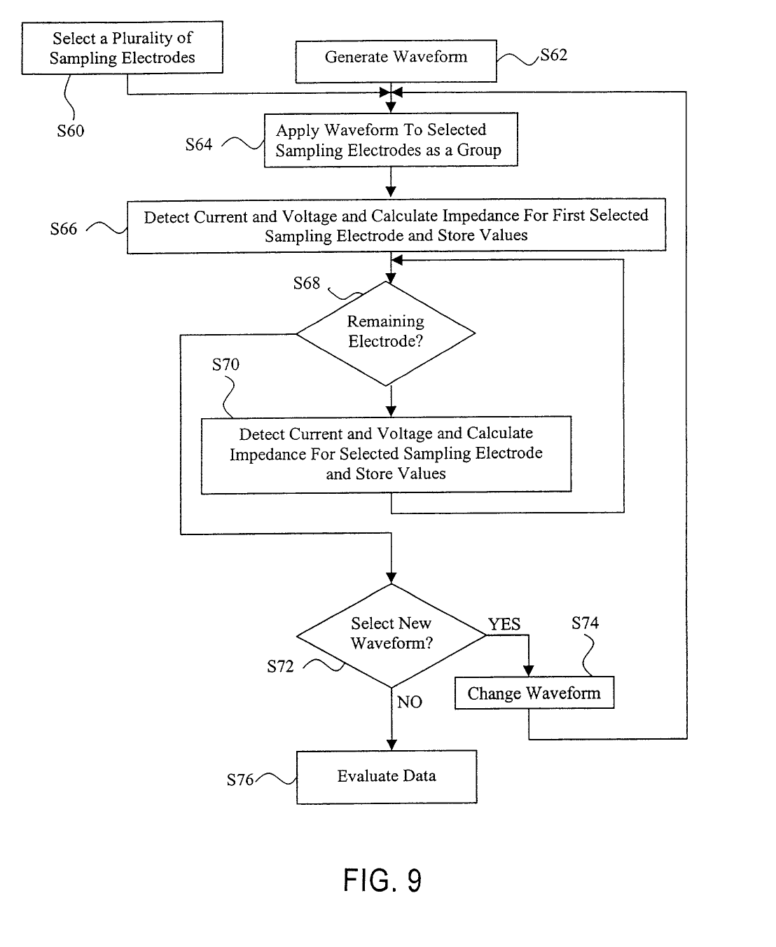

FIG. 9 illustrates yet another method of operating the apparatus of FIG. 5. As illustrated in FIG. 9, a plurality of sampling electrodes are selected (step S60) a generated waveform (step S62) is applied to the selected sampling electrodes as a group so that current of the generated waveform is distributed uniquely through each selected electrode (step S64). The current and voltage of each selected sampling electrode is detected and the impedance of each of the selected sampling electrodes is determined (steps S66, S68 and S70). Since each of the selected sampling electrodes are applied with a different current, and the voltage may vary between each of the sampling electrodes, both the current and voltage must be determined to calculate the impedance. Once the impedance is determined for the selected sampling electrodes (step S68, NO), the flow diagram determines if another waveform is to be selected and applied to the selected sampling electrodes and the data is evaluated in the same manner as done in the embodiment of FIG. 8 (steps S72, S74 and S76).

Although the embodiment of FIG. 5 has been described as detecting the current and voltage waveform at each sampling electrode to determine the impedance between each sampling electrode and the return electrode, those of skill in the art will appreciate that other techniques may be used. For example, one of the current or voltage waveforms could be detected at the sampling electrode while the other is detected at the return electrode, or both the voltage and the current waveforms may be detected at the return electrode.

The methods of FIGS. 6-9 are preferably executed or caused to be executed by the microprocessor. Instructions for performing the steps of the methods of FIGS. 6-9 may be stored on a computer readable medium. A computer readable medium is any tangible structure, such as a magnetic disk, an optical disk or a magnetic tape, or intangible structure, such as a modulated carrier wave containing packetized data, which is a wireline, optical cable or a wireless transmission, which is capable of being accessed by a microprocessor or computer.

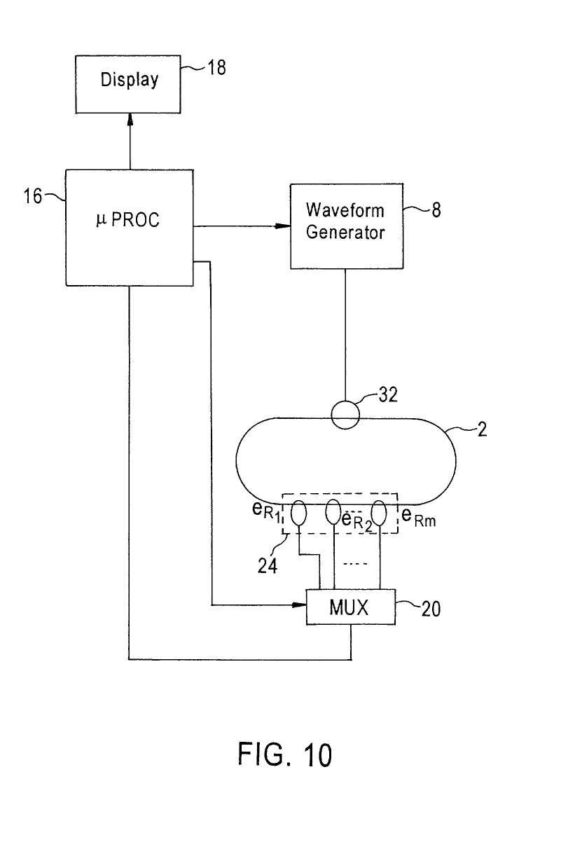

A second embodiment of the apparatus of the invention is illustrated in FIG. 10. The embodiment illustrated in FIG. 10 is similar to the embodiment illustrated in FIG. 5 except that a return electrode array 24 is used and a single sampling electrode 32 is used. As illustrated in FIG. 10, microprocessor 16 provides waveform generator 8 to provide sampling electrode 32 with a waveform. The return electrode array 24 contains a plurality of return electrodes e_{R1}through e_{Rm} which selectively complete an electrical circuit when selected by switching device 20 to provide a signal to the microprocessor. The impedance of the BERMS tissue is determined in the same manner as described in connection with the embodiment of FIG. 5, except that the current and voltage waveform may preferably be determined at the return electrodes instead of at the sampling electrode to allow for a more convenient broad area of coverage by the plurality of return electrodes. Those of skill in the art will appreciate that the methods of operating the apparatus of FIG. 5 depicted in FIGS. 6-9 are equally applicable to the embodiment of FIG. 10, except that the return electrodes are selected and that the waveform is applied to the return electrodes through the sampling electrode and the subject.

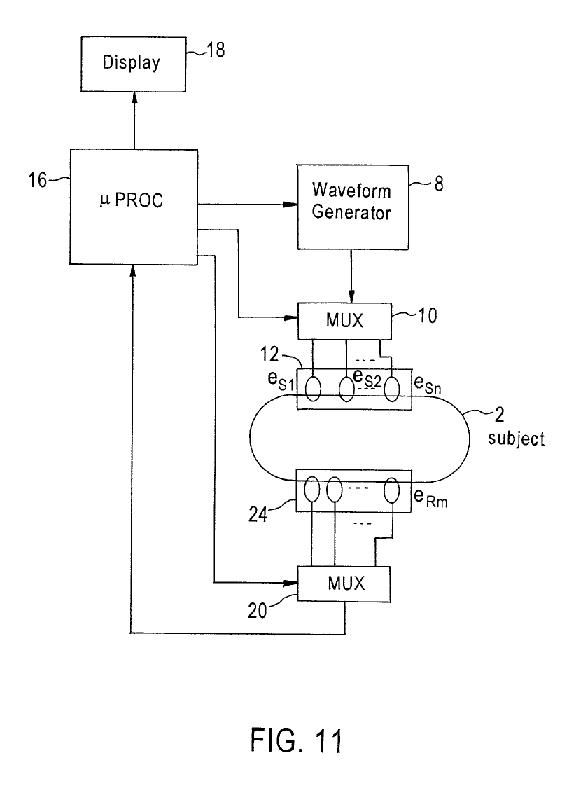

A third embodiment of the invention is illustrated in FIG. 11. The embodiment illustrated in FIG. 11 is a combination of the embodiments of FIG. 5 and FIG. 10. The embodiment of FIG. 11, includes both a sampling electrode array 12 and a return electrode array 24 and a second switching device 20. The return electrode array 24 also preferably contains a plurality of return electrodes eᵣ₁ through eₘ, where m may be any whole number and m may be equal to n, may less than n, or may be greater than n, where n is the number of sample electrodes in sample electrode array 12. The microprocessor 16 preferably controls both the switching device 10 and the switching device 24 to selectively control which sampling electrodes and which return electrodes are used for an impedance determination. Those of skill in the art will appreciate that the apparatus of the third embodiment in FIG. 11 may be operated in the same manner as described in FIGS. 6-9 with the additional selection of the desired return electrode(s) in return electrode array 24 which is/are used to complete the electrical circuit by switching device 20. Those of skill in the art will also appreciate that the embodiment of FIG. 11 may also be operated in the same manner as described in connect with the embodiment of FIG. 10, except that the sampling electrode in sampling electrode array 12 to be used to complete the electrical circuit may be selected by switching device 10.

Although a plurality of electrodes are illustrated in connection with the above described embodiments, those of skill in the art will appreciate that a single sampling electrode may used with a single return electrode. In this case, the methods of FIGS. 6-9 are equally applicable accept that a selection of electrodes is not needed.

The present invention may have many uses, including, for example, nerve avoidance, such as during placement of surgical trochars, or for the identification of abnormal tissue structures.

The present invention has many uses as will be readily appreciated by those of skill in the art. For example, without limitation, the present invention may be used to apply a mathematical analysis to the applied voltage data to extract information specific to nerve branching in a horizontal, vertical or oblique direction. The present invention may also be used to apply a mathematical analysis to the applied voltage data to extract information specific to nerve compression, nerve traction, nerve entrapment, nerve transection, or nerve contusion. The present invention may also be used to apply a mathematical analysis to applied voltage data to extract information specific to the presence of neuromas. The present invention may also be used to apply a mathematical analysis to applied voltage data to extract information specific to myofascial trigger points or to acupuncture points. The present invention may also be used to apply a mathematical analysis to applied voltage data to extract information specific to axonal demyelination. The present invention may also be used to apply a mathematical analysis to applied voltage data to extract information specific to normal nerve supplying pathological structures, such as joint, tendon, muscle, bone or other soft tissues. The present invention may also be used to allow targeting of specific therapies to nerve, such as injection of local anesthetic or botulinum toxin. The present invention may also be used to allow monitoring of nerve tissue over time for evaluation of the development of nerve abnormalities, such as carpal tunnel syndrome. The present invention may also be used to allow monitoring of nerve tissue over time for evaluation of the development of nerve abnormalities, such as pressure effects on nerves during surgery or other prolonged static positioning situations. The present invention may also be used to allow monitoring of nerve tissue over time for evaluation of nerve repair following neurolysis or neurorrhaphy or surgical repair of nerve transections. The present invention may also be used to allow targeting of other diagnostic studies, such as MRI, or electrodiagnostic studies, to specific nerves.

The foregoing description of the embodiments of the invention have been presented for purposes of illustration. It is not intended to be exhaustive or to limit the invention to the precise form disclosed, and obviously many modifications and variations are possible in light of the above disclosure.

What is claimed is:

1. An apparatus for detecting tissue structures comprising:
a microprocessor;
a waveform generator operable to generate a plurality of different periodic waveforms in response to instructions received from the microprocessor;
at least one sampling electrode operable to receive a waveform from the waveform generator and to apply the received waveform to a tissue of the subject as an applied waveform;
at least one return electrode operable to receive the applied waveform from the tissue of the subject and providing the applied waveform to the microprocessor, thereby completing an electrical circuit which includes the tissue of the subject as a component,
wherein the microprocessor receives information indicative of characteristics of the applied waveform and calculates a non-linear electrical characteristic of the tissue of the test subject.

2. The apparatus of claim 1, wherein the non-linear characteristic which is calculated is the impedance of the tissue.

3. The apparatus of claim 2, wherein the microprocessor is operable to: instruct the waveform generator to generate a plurality of different waveforms to be applied to the tissue, to selectively calculate the impedance of the tissue for each generated waveform of the plurality of different waveforms, and to perform mathematical calculations selectively using characteristics of the plurality of waveforms and the selectively calculated impedances of the tissue.

4. The apparatus of claim 3, wherein the mathematical calculation that is performed is a determination of a ratio of a change in impedance and a change in applied current.

5. The apparatus of claim 1, wherein the at least one sampling electrode comprises a plurality of sampling electrodes and wherein the apparatus further comprises a switching device operable to receive instructions from the microprocessor to provide a waveform to any sampling electrode of the plurality of sampling electrodes.

6. The apparatus of claim 5, wherein the switching device is operable to simultaneously provide a single waveform to more than one sampling electrode.

7. The apparatus of claim 5, wherein the switching device is operable to simultaneously provide a plurality of waveforms to more than one sampling electrode in a manner which provides the same current waveform to each of the sampling electrodes of the more than one sampling electrode.

8. The apparatus of claim 5, wherein the non-linear characteristic which is calculated is the impedance of the tissue.

9. The apparatus of claim 8, wherein the microprocessor is operable to: instruct the waveform generator to generate a plurality of different waveforms to be applied to the tissue, to selectively calculate the impedance of the tissue for each generated waveform of the plurality of different waveforms, and to perform mathematical calculations selectively using characteristics of the plurality of waveforms and the selectively calculated impedances of the tissue.

10. The apparatus of claim 9, wherein the mathematical calculation that is performed is a determination of a ratio of a change in impedance and a change in applied current.

11. The apparatus of claim 1, wherein the at least one return electrode comprises a plurality of return electrodes and wherein the apparatus further comprises a return switching device operable to receive instructions from the microprocessor to select any return electrode of the plurality of return electrodes to thereby complete an electrical circuit between the at least one sampling electrode and the selected return electrode.

12. The apparatus of claim 1, wherein the at least one sampling electrode comprises a plurality of sampling electrodes and wherein the apparatus further comprises a switching device operable to receive instructions from the microprocessor to provide a waveform to any sampling electrode of the plurality of sampling electrodes, and
wherein the at least one return electrode comprises a plurality of return electrodes and wherein the apparatus further comprises a return switching device operable to receive instructions from the microprocessor to select any return electrode of the plurality of return electrodes to thereby complete an electrical circuit between the at least one sampling electrode and the selected return electrode.

13. The apparatus of claim 1, wherein the non-linear characteristic which is calculated is the reactance of the tissue.

14. The apparatus of claim 1, further comprising a display, and wherein the microprocessor generates a three dimensional image of the tissue and the display is operable to display the three dimensional image.

15. A method of detecting tissue structures comprising the steps of:
generating a periodic waveform;
providing the periodic waveform to tissue of a subject through at least one sampling electrode as an applied waveform;
receiving the applied waveform from the tissue of the subject through at least one return electrode, thereby completing an electrical circuit which includes the tissue of the subject as a component,
receiving information indicative of the characteristic of the applied waveform; and
calculating a non-linear electrical characteristic of the tissue of the test subject associated with the applied waveform.

16. The method of claim 15, wherein the non-linear characteristic which is calculated is the impedance of the tissue.

17. The method of claim 15, further comprising the steps of:
generating a new periodic waveform which is different from a previous periodic waveform,
providing the new periodic waveform to the tissue of a subject through the sampling electrode as another applied waveform;
receiving the another applied waveform from the tissue of the subject through the return electrode, thereby completing an electrical circuit which includes the tissue of the subject as a component,
receiving information indicative of characteristics of the another applied waveform; and
recalculating a non-linear electrical characteristic of the tissue of the test subject associated with the another applied waveform.

18. The method of claim 17, wherein the non-linear electrical characteristic which is calculated is the impedance of the tissue, and the recalculated non-linear electrical characteristic is the impedance of the tissue, further comprising the step of
performing mathematical calculations selectively using characteristics of the another applied waveform and characteristics of the applied waveform and the calculated impedance of the tissue and the recalculated impedance of the tissue.

19. The method of claim 18, wherein the mathematical calculation that is performed is a determination of a ratio of a change in impedance and a change in applied current.

20. The method of claim 15, wherein the at least one sampling electrode comprises a plurality of sampling electrodes, and wherein the method further comprises the step of:
simultaneously providing a single waveform to more than one sampling electrode.

21. The method of claim 20, further comprising the steps of:
generating a new periodic waveform which is different from a previous periodic waveform,
providing the new periodic waveform to the tissue of a subject through the sampling electrode as another applied waveform;
receiving the another applied waveform from the tissue of the subject through the return electrode, thereby completing an electrical circuit which includes the tissue of the subject as a component,
receiving information indicative of characteristics the another applied waveform; and
recalculating a non-linear electrical characteristic of the tissue of the test subject associated with the another applied waveform.

22. The method of claim 21, wherein the non-linear electrical characteristic which is calculated is the impedance of the tissue, and the recalculated non-linear electrical characteristic is the impedance of the tissue, further comprising the step of
performing mathematical calculations selectively using characteristics of the another applied waveform and characteristics of the applied waveform and the calculated impedance of the tissue and the recalculated impedance of the tissue.

23. The method of claim 22, wherein the mathematical calculation that is performed is a determination of a ratio of a change in impedance and a change in applied current.

24. The method of claim 15, wherein the at least one sampling electrode comprises a plurality of sampling electrodes, and wherein the method further comprises the step of:
simultaneously providing a plurality of waveforms to more than one sampling electrode in a manner which provides the same current waveform to each of the sampling electrodes of the more than one sampling electrode.

25. The method of claim 24, further comprising the steps of:
generating a new periodic waveform which is different from a previous periodic waveform,
providing the new periodic waveform to the tissue of a subject through the sampling electrode as another applied waveform;
receiving the another applied waveform from the tissue of the subject through the return electrode, thereby completing an electrical circuit which includes the tissue of the subject as a component,
receiving information indicative of the voltage and current of the another applied waveform; and
recalculating a non-linear electrical characteristic of the tissue of the test subject associated with the another applied waveform.

26. The method of claim 25, wherein the non-linear electrical characteristic which is calculated is the impedance of the tissue, and the recalculated non-linear electrical characteristic is the impedance of the tissue, further comprising the step of
performing mathematical calculations selectively using characteristics of the another applied waveform and characteristics of the applied waveform and the calculated impedance of the tissue and the recalculated impedance of the tissue.

27. The method of claim 26, wherein the mathematical calculation that is performed is a determination of a ratio of a change in impedance and a change in applied current.

28. The method of claim 15, wherein the at least one return electrode comprises a plurality of return electrodes and wherein the method further comprises the step of:
selecting at least one return electrode of the plurality of return electrodes to thereby complete an electrical circuit between the at least one sampling electrode and the at least one selected return electrode.

29. The method of claim 15, wherein the at least one sampling electrode comprises a plurality of sampling electrodes and the at least one return electrode comprises a plurality of return electrodes, and wherein the method further comprises the steps of:
selecting at least one sampling electrode through which the periodic waveform is applied to the tissue of a subject as an applied waveform;
selecting at least one return electrode of the plurality of return electrodes to thereby complete an electrical circuit between the at least one sampling electrode and the at least one selected return electrode.

30. The method of claim 15, wherein the non-linear characteristic which is calculated is the reactance of the tissue.

31. The method of claim 15, further comprising the steps of:
generating a three dimensional image display of the tissue; and
displaying the three dimensional image.

32. A computer readable medium carrying instructions to cause a computer to institute the performance of a method, the method comprising the steps of:
generating a periodic waveform;
providing the periodic waveform to tissue of a subject through at least one sampling electrode as an applied waveform;
receiving the applied waveform from the tissue of the subject through at least one return electrode, thereby completing an electrical circuit which includes the tissue of the subject as a component,
receiving information indicative of characteristics of the applied waveform; and
calculating a non-linear electrical characteristic of the tissue of the test subject associated with the applied waveform.

33. The computer readable medium of claim 32, wherein the non-linear characteristic which is calculated is the impedance of the tissue.

34. The computer readable medium of claim 32, further containing instructions to cause a computer to institute performance of a method further comprising the steps of:
generating a new periodic waveform which is different from a previous periodic waveform,
providing the new periodic waveform to the tissue of a subject through the sampling electrode as another applied waveform;
receiving the another applied waveform from the tissue of the subject through the return electrode, thereby completing an electrical circuit which includes the tissue of the subject as a component,
receiving information indicative of the voltage and current of the another applied waveform; and
recalculating a non-linear electrical characteristic of the tissue of the test subject associated with the another applied waveform.

35. The computer readable medium of claim 32, wherein the non-linear electrical characteristic which is calculated is the impedance of the tissue, and the recalculated non-linear electrical characteristic is the impedance of the tissue, the computer readable medium further containing instructions to cause a computer to institute performance of a method further comprising the steps of:
performing mathematical calculations selectively using characteristics of the another applied waveform and characteristics of the applied waveform and the calculated impedance of the tissue and the recalculated impedance of the tissue.

36. The computer readable medium of claim 35, wherein the mathematical calculation that is performed is a determination of a ratio of a change in impedance and a change in applied current.

37. The computer readable medium of claim 32, wherein the at least one sampling electrode comprises a plurality of sampling electrodes, and wherein the computer readable medium further contains instructions to cause a computer to perform a method further comprising the step of:
simultaneously providing a single waveform to more than one sampling electrode.

38. The computer readable medium of claim 37, wherein the computer readable medium further contains instructions to cause a computer to institute performance of a method further comprising the steps of:
generating a new periodic waveform which is different from a previous periodic waveform,
providing the new periodic waveform to the tissue of a subject through the sampling electrode as another applied waveform;
receiving the another applied waveform from the tissue of the subject through the return electrode, thereby completing an electrical circuit which includes the tissue of the subject as a component,
receiving information indicative of the voltage and current of the another applied waveform; and
recalculating a non-linear electrical characteristic of the tissue of the test subject associated with the another applied waveform.

39. The computer readable medium of claim 38, wherein the non-linear electrical characteristic which is calculated is the impedance of the tissue, and the recalculated non-linear electrical characteristic is the impedance of the tissue, the computer readable medium further containing instructions to cause a computer to institute performance of a method further comprising the steps of:
performing mathematical calculations selectively using characteristics of the another applied waveform and characteristics of the applied waveform and the calculated impedance of the tissue and the recalculated impedance of the tissue.

40. The computer readable medium of claim 39, wherein the mathematical calculation that is performed is a determination of a ratio of a change in impedance and a change in applied current.

41. The computer readable medium of claim 32, wherein the at least one sampling electrode comprises a plurality of sampling electrodes, and wherein the computer readable medium further contains instructions to cause a computer to institute performance of a method further comprising the steps of:
simultaneously providing a plurality of waveforms to more than one sampling electrode in a manner which provides the same current waveform to each of the sampling electrodes of the more than one sampling electrode.

42. The computer readable medium of claim 41, wherein the computer readable medium further contains instructions to cause a computer to institute performance of a method further comprising the steps of:
generating a new periodic waveform which is different from a previous periodic waveform,
providing the new periodic waveform to the tissue of a subject through the sampling electrode as another applied waveform;
receiving the another applied waveform from the tissue of the subject through the return electrode, thereby completing an electrical circuit which includes the tissue of the subject as a component,
receiving information indicative of the voltage and current of the another applied waveform; and
recalculating a non-linear electrical characteristic of the tissue of the test subject associated with the another applied waveform.

43. The computer readable medium of claim 42, wherein the non-linear electrical characteristic which is calculated is the impedance of the tissue, and the recalculated non-linear electrical characteristic is the impedance of the tissue, the computer readable medium further containing instructions to cause a computer to institute performance of a method further comprising the steps of:
performing mathematical calculations selectively using characteristics of the another applied waveform and characteristics of the applied waveform and the calculated impedance of the tissue and the recalculated impedance of the tissue.

44. The computer readable medium of claim 43, wherein the mathematical calculation that is performed is a determination of a ratio of a change in impedance and a change in applied current.

45. The computer readable medium of claim 32, wherein the at least one return electrode comprises a plurality of return electrodes and wherein the computer readable medium further contains instructions to cause a computer to institute performance of a method further comprising the step of:
selecting at least one return electrode of the plurality of return electrodes to thereby complete an electrical circuit between the at least one sampling electrode and the at least one selected return electrode.

46. The computer readable medium of claim 32, wherein the at least one sampling electrode comprises a plurality of sampling electrodes and the at least one return electrode comprises a plurality of return electrodes, and wherein the computer readable medium further contains instructions to cause a computer to institute performance of a method further comprising the steps of:
selecting at least one sampling electrode through which the periodic waveform is applied to the tissue of a subject as an applied waveform;
selecting at least one return electrode of the plurality of return electrodes to thereby complete an electrical circuit between the at least one sampling electrode and the at least one selected return electrode.

47. The computer readable medium of claim 32, wherein the non-linear characteristic which is calculated is the reactance of the tissue.

48. The computer readable medium of claim 47, wherein the computer readable medium further contains instructions to cause a computer to institute performance of a method further comprising the steps of:
generating a three dimensional image display of the tissue; and
displaying the three dimensional image.
